SEQUENCE LISTING (2 sequences)

SEQ ID NO: 1 - DNA, 6085 bp, Artificial Sequence
  Expression Plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttaattaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaaattcaggaggttgctctaatgatgattactctgcgcaaacttcctctggcggttgccgtcgcagcgggcgtaatgtctgctcaggcaatggctgttgatttccacggctatgcacgttccggtattggttggacaggtagcggcggtgaacaacagtgtttccagactaccggtgctcaaagtaaataccgtcttggcaacgaatgtgaaacttatgctgaattaaaattgggtcaggaagtgtggaaagagggcgataagagcttctatttcgacactaacgtggcctattccgtcgcacaacagaatgactgggaagctaccgatccggccttccgtgaagcaaacgtgcagggtaaaaacctgatcgaatggctgccaggctccaccatctgggcaggtaagcgcttctaccaacgtcatgacgttcatatgatcgacttctactactgggatatttctggtcctggtgccggtctcgagtctagactggaaaacatcgatgttggcttcggtaaactctctctggcagcaacccgctcctctgaagctggtggttcttcctctttcgccagcaacaatatttatgactataccaacgaaaccgcgaacgacgttttcgatgtgcgtttagcgcagatggaaatcaacccgggcggcacattagaactgggtgtcgactacggtcgtgccaacttgcgtgataactatcgtctggttgatggcgcatcgaaagacggctggttattcactgctgaacatactcagagtgtcctgaagggctttaacaagtttgttgttcagtacgctactgactcgatgacctcgcagggtaaagggctgtcgcagggttctggcgttgcatttgataacgaaaaatttgcctacaatatcaacaacaacggtcacatgctgcgtatcctcgaccacggtgcgatctccatgggcgacaactgggacatgatgtacgtgggtatgtaccaggatatcaactgggataacgacaacggcaccaagtggtggaccgtcggtattcgcccgatgtacaagtggacgccaatcatgagcaccgtgatggaaatcggctacgacaacgtcgaatcccagcgcaccggcgacaagaacaatcagtacaaaattaccctcgcacaacaatggcaggctggcgacagcatctggtcacgcccggctattcgtgtcttcgcaacctacgccaagtgggatgagaaatggggttacgactacaccggtaacgctgataacaacgcgaacttcggcaaagccgttcctgctgatttcaacggcggcagcttcggtcgtggcgacagcgacgagtggaccttcggtgcccagatggaaatctggtggtaaatccccgcgccctcatccgaaagggcgtattggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 4812 bp, Artificial Sequence
  Expression Plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttaattaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaaattcaggaggttgctctaatgaacttggggaatcgactgtttattctgatagcggtcttacttcccctcgcagtattactgctcgtcgactctagaggatccccgcgccctcatccgaaagggcgtattggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2009/0004744 A1
(43) Pub. Date: Jan. 1, 2009

(54) MINICELLS AS VACCINES

(75) Inventors: Mark W. Surber, San Diego, CA (US); Roger Sabbadini, Lakeside, CA (US); Neil Berkley, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: Vaxiion Therapeutics, Inc., San Diego, CA (US)

(21) Appl. No.: 12/113,169

(22) Filed: Apr. 30, 2008

Related U.S. Application Data
(63) Continuation of application No. 11/096,646, filed on Apr. 1, 2005, now abandoned.
(60) Provisional application No. 60/559,820, filed on Apr. 5, 2004.

(51) Int. Cl. C12N 15/75; C12N 15/74

(52) U.S. Cl. 435/476; 435/252.3; 435/252.31

(57) ABSTRACT

The disclosed invention relates to immunogenic minicells cells (anucleated) and their use to induce an immune response from a subject.

### RELATED APPLICATIONS

This application is a continuation of U.S. application Ser. No. 11/096,646, filed Apr. 1, 2005, which claims the benefit of U.S. Provisional Application No. 60/559,820, filed Apr. 5, 2004, both of which are incorporated herein by reference in its entirety.

### FIELD OF THE INVENTION

The disclosed invention relates to immunogenic minicells and their use to induce an immune response from a subject.

### BACKGROUND OF THE INVENTION

A variety of recombinant protein expression systems have been used to produce immunogenic compositions. Some commonly used expression systems include the rabbit reticulocyte lysate system, E. coli S30 Extract System (both available from PROMEGA) (Zubay, Methods Enz. 65:856, 1980), eukaryotic cell culture expression, and bacterial expression systems.

Bacterial expression systems are generally similar to that of the eukaryotic expression systems in that they both use the host cell enzymes to drive protein expression from recombinant expression vectors. In bacterial expression systems, bacterial cells are transformed with expression elements from which transcription is driven. The resulting messenger ribonucleic acid (mRNA) is translated by the host cell, thus yielding a protein of interest.

Bacteria divide very rapidly and are easy to culture; it is relatively easy to produce a large number of bacteria in a short time. Moreover, incorporation of expression elements into bacterial cells is efficient. Cultures of transformed cells can be grown to be genetically identical. Thus, all cells in the culture will contain the expression element.

Bacterial expression systems can be used to produce membrane proteins for use in immunogenic compositions. Although bacterial expression systems can be used to produce antigenic material, there are a variety of disadvantages to use such a system. For example, the potential for contamination of the immunogenic product with live, reproducing bacterial cells renders bacterial expression systems undesirable for producing immunogenic material. Similar drawbacks exist when immunogenic compositions are prepared using eukaryotic host cells.

Minicells produced by host cells are advantageous over whole-cell protein expression systems. Using minicells to produce antigenic compositions greatly reduced the likelihood of contamination with a whole, live cell. Khachatourians (U.S. Pat. No. 4,311,797) exploited an E. coli strain that constitutively produced anucleated minicells and constitutively expressed the K99 surface antigen. The resulting E. Coli derived minicells were prepared as a vaccine. The vaccine induced the production of antibodies against growing and infective enteropathogenic K99+ E. coli in cattle and was, thus effective against coliform enteritis. It is important to note that this reference only teaches the use of E. coli based minicells to express a naturally occurring E. coli gene. Accordingly, there is still a need in the art to produce immunogenic minicells capable of expressing heterologous genes to stimulate an immunogenic response in a subject.

### SUMMARY OF THE INVENTION

Disclosed herein is a method of preparing an immunogenic minicell including preparing an inducible expression vector, wherein the inducible expression vector comprises a heterologous nucleotide sequence encoding an open reading frame of an antigen of interest; introducing the expression vector to an inducible minicell producing parent cell; inducing minicell formation from the minicell producing parent cell; inducing expression of the open reading from of the antigen of interest; and purifying minicells from the inducible minicell producing parent cell. In one embodiment, the expression vector comprises the heterologous nucleotide sequence encoding the open reading frame of an antigen of interest operably linked to a nucleotide sequence encoding a transmembrane protein. In one embodiment, the open reading frame of an antigen of interest encodes a transmembrane protein. In one embodiment, the transmembrane protein is expressed on the outer membrane of the minicell. In one embodiment, the transmembrane protein is expressed on the inner membrane of the minicell. In one embodiment, the immunogenic minicell is derived from a Gram-negative bacterial parent cell. In one embodiment, the Gram-negative bacteria is selected from the group consisting of Campylobacter jejuni, S. dysenteriae, Lactobacillus spp., Neisseria gonorrhoeae, Legionella Pneumophila, Salmonella spp., Shigella flexneri, and Escherichia coli. In one embodiment, the immunogenic minicell is derived from a Gram-positive bacterial parent cell. In one embodiment, the Gram-positive bacteria is selected from the group consisting of Staphylococcus spp., Streptococcus spp., Bacillus subtilis and Bacillus cereus. In one embodiment, the open reading frame of the antigen of interest is derived from a Bacillus anthracis genome. In one embodiment, the open reading frame of the antigen of interest encodes a protective antigen with an accession number selected from the group consisting of NC_{—}001496, NC_{—}003980, AF306783, AF306782, AF306781, AF306780, and AF306779. In one embodiment, the open reading frame of the antigen of interest is derived from a Clostridium botulinum genome. In one embodiment, the open reading frame of the antigen of interest encodes a protective antigen with an accession number selected from the group consisting of M27892, AY166872, AF464912, AF295926, AF300469, AF300468, AF300467, AF300466, AF300465, D49440, X62389, D90210, D88982, D63903, X54254, AB082519, X70815, X70818, X62683, X62089, Y10770, X70821, X70820, X70816, M92906, AX608812, and X74162. In one embodiment, the open reading frame of the antigen of interest is derived from a Yersinia pestis genome. In one embodiment, the open reading frame of the antigen of interest encodes a protective antigen with an accession number selected from the group consisting of X61996, AF053945, NC_{—}003131, AF167310, AF167309, NC_{—}003131, AF074612, and AF053946.

Also disclosed herein is a eubacterial minicell including a heterologous antigen of interest wherein the antigen of interest is derived from a genome of a pathogen selected from the group consisting of Bacillus anthracis (anthrax), Clostridium botulinum (Botulism), Yersinia pestis, Variola major (smallpox), Francisella tularensis (tularemia), LCM virus, junin virus, machup virus, guanarito virus, lassa fever virus, bunyavirus, hantaviruse, rift valley fever virus, dengue virus, ebola virus, and marburg virus. In one embodiment, the transmembrane protein is expressed on the outer membrane of the minicell.

In one embodiment, the transmembrane protein is expressed on the inner membrane of the minicell. In one embodiment, the immunogenic minicell is derived from a Gram-negative bacterial parent cell. In one embodiment, the Gram-negative bacteria is selected from the group consisting of Campylobacter jejuni, S. dysenteriae, Lactobacillus spp., Neisseria gonorrhoeae, Legionella Pneumophila, Salmonella spp., Shigella flexneri, and Escherichia coli. In one embodiment, the immunogenic minicell is derived from a Gram-positive bacterial parent cell. In one embodiment, the Gram-positive bacteria is selected from the group consisting of Staphylococcus spp., Streptococcus spp., Bacillus subtilis and Bacillus cereus. In one embodiment, the open reading frame of the antigen of interest is derived from a Bacillus anthracis genome. In one embodiment, the antigen of interest is encoded by a polynucleotide having an accession number selected from the group consisting of NC_{—}001496, NC_{—}003980, AF306783, AF306782, AF306781, AF306780, and AF306779. In one embodiment, the open reading frame of the antigen of interest is derived from a Clostridium botulinum genome. In one embodiment, the open reading frame of the antigen of interest encodes a protective antigen with an accession number selected from the group consisting of M27892, AY166872, AF464912, AF295926, AF300469, AF300468, AF300467, AF300466, AF300465, D49440, X62389, D90210, D88982, D63903, X54254, AB082519, X70815, X70818, X62683, X62089, Y10770, X70821, X70820, X70816, M92906, AX608812, and X74162. In one embodiment, the open reading frame of the antigen of interest is derived from a Yersinia pestis genome. In one embodiment, the open reading frame of the antigen of interest encodes a protective antigen with an accession number selected from the group consisting of X61996, AF053945, NC_{—}003131, AF167310, AF167309, NC_{—}003131, AF074612, and AF053946.

### DETAILED DESCRIPTION OF THE PREFERRED EMBODIMENT

The disclosed invention relates to the use of minicells for the preparation of immunogenic material. The applications of immunogenic minicells include research, prophylactic, diagnostic and therapeutic applications. The National Institute for Allergy and Infectious Diseases has categorized pathogenic targets for research into three groups, Category A pathogens, Category B pathogens, and Category C pathogens. Any antigen from Categories A, B, and C pathogens can be used with the immunogenic minicells described herein. As an example, the pathogens of Category A, discussed below, provide a number of relevant antigens that can be displayed upon the surface of a minicell in order to use the resulting material as an immunogenic composition effective in protecting subjects from infection of these agents.

In a preferred embodiment, the immunogenic minicells provided herein encode and are capable of expressing a heterologous antigenic product. Accordingly, in more specific embodiments the described minicells can include a vector having a heterologous nucleotide sequence encoding an open reading frame of an antigen of interest. In even more particular embodiments, the immunogenic minicells described herein include vectors having an open reading frame encoding a cancer derived or pathogen derived antigen. As used herein, the term “heterologous” relates to an antigen encoded by the genome of a species other than that from which the minicell is derived from. Antigens can be encoded by any suitable species including pathogens, such as viruses, bacteria, fingi, protozoa, and the like. Antigens can also be encoded by human beings and other mammals. This embodiment is particularly advantageous when it is desirable to have minicells displaying a cancer antigen.

Immunogenic Minicells

Minicells can be used to immunize subjects. An organism is “immunized” when it is contacted with an immunogen and the organism produces an immune response to the immunogen. The immune response can be protective or therapeutic. Examples of protective or therapeutic immune responses include the generation of antibodies, such as neutralizing antibodies, or engendering the proliferation or activity of cytotoxic cells against the immunogen.

Immunization strategies can be divided generally into two classes: the use of whole-killed or attenuated pathogens to present immunogens and the isolation of immunogens from a pathogen for use as an immunogen. Presentation of whole-killed or attenuated pathogens has many advantages and frequently produces a robust immune response from an immunized organism.

The use of whole-killed or attenuated pathogens as immunogens, however, has certain risks. Perhaps the most serious of these risks involves the possibility the attenuated immunogen is still sufficiently viable to cause disease in an organism immunized with the attenuated immunogen. Accidental infections with various polio and smallpox vaccines are just two examples of this type of risk.

With the advent of molecular biology techniques it became possible to isolate particular antigens from a pathogen for use in an immunogenic composition. Often these immunogenic compositions comprise a subunit of a pathogen that, when presented to an organism, will permit the immunized organism to generate a protective immune response. One extremely successful example of such a subunit immunogen is the hepatitis B subunit vaccine.

Using an immunogenic composition comprising a subunit of a pathogen is advantageous as it reduces the risk of accidental infection. Unfortunately, isolating one or more subunits from a pathogen for use in an immunogenic composition often leads to a weaker immunogenic composition when compared to a whole-killed or attenuated immunogen. One possible explanation for this phenomenon holds that the isolated and preformed antigenic subunit is altered during production and is no longer in its native confirmation. The resulting immune response to the misshapen immunogen does not serve to prepare a host's immune system to raise a protective immune response against the pathogen of interest.

The use of minicells to present antigens for immunization has several potential advantages. For example, immunogenic minicells are able to present one or more antigenic membrane proteins in their native form to a host's immune system. Native presentation of antigens can be superior to presenting antigens in a non-native form because the immune system response is more likely to recognize an active pathogen based on the prior exposure to the immunogenic minicells.

In addition to the presentation of antigens in their native conformation, immunogenic minicells may present antigens in a manner that more accurately mimics antigen presentation by the active pathogen from which the antigen of interest was derived. For example, most non-enveloped viral pathogens present one or more antigenic epitopes on the surface of their viral coats in a repeating format. It has been theorized that mammalian immune systems have adapted to recognize the presence of repeating antigenic epitopes as the hallmark of an invading viral pathogen. Immunogenic minicells displaying one or more antigens on their surface may be able to more accurately mimic the antigen presentation of a native virus particle and thus elicit a more robust immune response than merely providing an antigen to a host in a non-structured format.

Immunogenic minicells have other advantages over standard immunogenic compositions. Specifically, minicell producing parent cells lines often contain immunogenic components, even in the absence of an antigen of interest being introduced. For example, the lipopolysaccharide component of Gram-negative bacteria is known to be extremely immunogenic. Immunogenic minicells displaying an antigen of interest can possibly elicit a more robust immune response from a host than that elicited by a purified, performed antigen because the host may recognize various components of the minicell as a foreign antigen. As such, immunogenic minicells present both an antigen of interest and an adjuvant to the immune system of a host organism. In addition to native minicell components that may act as adjuvants, the immunogenic minicells disclosed herein may also be altered to include non-native adjuvant molecules that further increase the immunogenicity of the minicell compositions.

In research applications, immunogenic minicells can be used to generate antibodies to an antigen displayed by a minicell. Such antibodies can be used to detect an antigen, which may be a chemical moiety, molecule, virus, organelle, cell, tissue, organ, or organism that one wishes to study. Classically, such antibodies have been prepared by immunizing an animal, often a rat or a rabbit, and collecting antisera therefrom. Molecular biology techniques can be used to prepare antibodies and antibody fragments, as is described elsewhere herein. Single-chain antibody fragments (scFv) may also be identified, purified, and characterized using minicells displaying a membrane protein or membrane bound chimeric soluble protein.

In prophylactic applications, immunogenic minicells are used to stimulate an immune response from a subject. After administration of the immunogenic composition disclosed herein, the subject is “pre-immunized” to a pathogen before contact with the pathogen occurs. This pre-immunization allows the subject to mount a protective immune response to the particular pathogen, thus preventing disease in the subject.

Certain aspects of the invention involve active immunotherapy. Active immunotherapy relies on the in vivo stimulation with an immunogenic composition of the endogenous host immune system. Exemplary immunogenic compositions include immunogens, allergens, toxins, adjuvants, cytokines and chemokines, all of which allow the host immune system to react against pathogens.

Other therapeutic applications involve passive immunotherapy. Passive immunotherapy involves the administration of agents (such as antibodies or effector cells) directed against an immunogen of a pathogen. Passive immunotherapy does not necessarily depend on an intact host immune system. Examples of effector cells include T cells; T lymphocytes, such as CD8+ cytotoxic T lymphocytes and CD4+ T-helper tumor-infiltrating lymphocytes; killer cells, such as Natural Killer (NK) cells and lymphokine-activated killer cells.

Minicell Production

Minicells are anucleated cells that lack chromosomal DNA derived from the minicell producing parent cells from which they are produced. The term “minicells” encompasses derivatives of eubacterial and archaebacterial cells that lack parental chromosomal DNA as well as anucleated derivatives of eukaryotic cells. The immunogenic minicells described herein can be derived from both gram-positive and gram-negative parental cells.

Minicells are produced by minicell producing parent cells. These parent cells undergo cell division in an abnormal manner that produces a chromosomal-containing cell and a minicell lacking a copy of the parental chromosome. Minicells are often smaller than their parent cells. For example, minicells produced from E. coli cells are generally spherical in shape and are about 0.1 to about 0.3 um in diameter, whereas whole E. coli cells are about from about 1 to about 3 um in diameter and from about 2 to about 10 um in length. Table 1 shows a variety of minicell-producing sources and discusses the mechanisms by which the minicells are generated.

TABLE 1
Eubacterial Strains, Mutations and Conditions
that Promote Minicell Formation

| Species | Strain | Notes | References |
|---|---|---|---|
| Campylobacter jejuni | | May occur naturally late in | Brock et al., 1987 |
| | | growth cycle | |
| Bacillus subtilis | | Mutations in divIVB locus | Barak et al., 1999 |
| | | (inc. minC, minD | |
| | | ripX mutations | Sciochetti et al., |
| | | | 1999; Lemon et al., |
| | | | 2001 |
| | | smc mutations | Moriya et al., 1998; |
| | | | Britton et al., 1998 |
| | | oriC deletions | Moriya et al., 1997; |
| | | | Hassan et al., 1997 |
| | | prfA mutations | Pederson and |
| | | | Setlow, 2001 |
| | | Mutations in divIVA locus | Cha et al., 1997 |
| | B.s. 168 | ts initiation mutation TsB143 | Sargent, 1975 |
| Bacillus cereus | WSBC | Induced by exposure to long- | Maier et al., 1999 |
| | 10030 | chain polyphosphate | |
| Shigella flexneri (2a) | MC-1 | | Gemski et al., 1980 |
| S. dysenteriae (1) | MC-V | | Gemski et al., 1980 |
| Lactobacillus spp. | | Variant minicell-producing | Pidoux et al., 1990 |
| | | strains isolated from grains | |
| Neisseria | | deletion or overepression of | Ramirez-Arcos et |
| gonorrhoeae | | min homologues | al., 2001; Szeto et |
| | | | al., 2001 |
| Escherichia coli | | MinA mutations | Frazer et al., 1975; |
| | | | Cohen et al. 1976 |
| | | MinB mutations and deletions | Adler et al., 1967; |
| | | | Davie et al., 1984; |
| | | | Schaumberg et al.; |
| | | | 1983; Jaffe et al., |
| | | | 1988; Akerlund et |
| | | | al., 1992 |
| | CA8000 | cya, crp mutations | Kumar et al.; 1979 |
| | | MukA1 mutation | Hiraga et al., 1996 |
| | | MukE, mukF mutations | Yamanaka et al., |
| | | | 1996 |
| | | hns mutation | Kaidow et al., 1995 |
| | DS410 | | Heighway et al., |
| | | | 1989 |
| | | χ1972, χ 1776 and χ 2076 | Curtiss, 1980 |
| | P678-54 | Temperature-sensitive cell | Adler et al. 1967; |
| | | division mutations | Allen et al., 1972; |
| | | | Hollenberg et al., |
| | | | 1976 |
| | | Induced by overexpression of | De Boer et al., 1988 |
| | | minB protein | |
| | | Induced by overexpression of | Pichoff et al., 1995 |
| | | minE protein or derivatives | |
| | | Induced by overproduction of | Ward et al., 1985 |
| | | ftsZ gene | |
| | | Induced by overexpression of | Wang et al., 1991 |
| | | sdiA gene | |
| | | Induced by overexpression of | Ramirez-Arcos et |
| | | min genes from Neisseria | al., 2001; Szeto et |
| | | gonorrhoeae | al., 2001 |
| | | Induced by exposure to EGTA | Wachi et al., 1999 |
| Legionella | | Induced by exposure to | Elliot et al., 1985 |
| pneumophila | | ampicillin | |

Minicells are produced by several different eubacterial strains and mechanisms including the overexpression of endogenous or exogenous genes involved in cell division, chromosomal replication and partitioning, mutations in such genes, and exposure to various chemical and/or physical conditions. For example, E. coli cells that overexpress the gene product FtsZ, a protein involved in the regulation of cell division, form minicells. Minicells are also produced by E. coli cells having a mutation in one or more genes of the min locus, which is a group of genes that encode proteins that are involved in cell division.

Eubacterial cells that have been shown to produce minicells include Escherichia, Shigella, Bacillus, Lactobacillus, Salmonella, Legionella and Campylobacter. Bacterial minicell-producing species of particular interest are E. coli, Salmonella spp., and Bacillus subtilis. These organisms are particularly amenable to manipulation by a variety of molecular and genetic methods, with a variety of well-characterized expression systems, including many episomal and chromosomal expression systems, as well as other factors and elements useful in the present invention.

Because minicells lack the chromosomal DNA of the parent cell, RNA and protein production in the minicells is dependent on the protein production components that segregate into the minicell during formation. It has been alternatively reported that few molecules of endogenous RNA polymerase segregate into minicells and that many RNA polymerase molecules follow plasmids into minicells. Introduction of an exogenous RNA polymerase to minicell-producing cells enhances expression of episomal elements in minicells. Such enhanced expression may allow for the successful expression of proteins in minicells, wherein such proteins are expressed poorly or not at all in unmodified minicells. In order to maximize the amount of RNA transcription from episomal elements in minicells, minicell-producing cell lines that express an RNA polymerase specific for certain episomal expression elements may be used. An example of an E. coli strain of this type, designated MC-T7, was created and used as is described in the Examples. Those skilled in the art will be able to make and use equivalent strains based on the present disclosure and their knowledge of the art. Minicell construction is discussed in more detail in U.S. Publication No. 2003-0194798, filed May 24, 2003, which is hereby incorporated by reference in its entirety.

Production of minicells and protein production therefrom may occur using a variety of approaches or combination thereof. In one approach, minicells are formed and purified. Expression elements contained in the minicells are then stimulated or induced to produce gene products encoded by the expression elements. In another approach, minicell-producing parent cells containing one or more expression elements are stimulated or induced to express a protein of interest. Minicell production is subsequently induced in this approach. In yet another approach, minicell production and protein production are co-induced. The disclosed methods of minicell production teach the exploitation of any timing variable of minicell formation or protein production to optimize minicell and protein production

It is desirable to optimize minicell and protein production from minicell producing parent cells because these functions can be detrimental to the host cells. Using inducible minicell and protein production systems permits one to minimize the deleterious effects of these procedures. For example, an inducible promoter can be used to control the expression of one or more genes that induce minicell formation. The same inducible promoter or a different inducible promoter can be used to control protein expression from the minicells. Yields of immunogenic minicells can be optimized by timing the induction of minicell production from a minicell producing parent cell line with induction of protein production from one or more expression vectors encapsulated within the minicell.

Minicell Purification

A variety of methods are used to separate minicells from parent cells. In general, such methods are physical, biochemical and genetic, and can be used in combination. The objective of these methods is to minimize or eliminate parental cell contamination in the minicell compositions produced using the described methods. For example, minicells are separated from parent cells using glass-fiber filtration and centrifugation, both differential and zonal, size-exclusion chromatography, differential sonication, and freeze-thaw cycles.

One centrifugation technique provides for the purification of minicells can be purified using a double sucrose gradient. The first centrifugation involves differential centrifugation, which separates parent cells from minicells based on differences in size or density. The percent of sucrose in the gradient (graduated from about 5 to about 20%), Ficol or glycerol is designed to allow only parent cells to pass through the gradient.

The supernatant, which is enriched for minicells, is then separated from the pellet and is spun at a much higher rate (for example, ≧11,000×g). This pellets the minicells and any parent cells that did not pellet out in the first spin. The pellet is then resuspended and layered on a sucrose gradient.

The band containing minicells is collected, pelleted by centrifugation, and loaded on another gradient. This procedure is repeated until the minicell preparation is essentially depleted of parent cells, or has a concentration of parent cells that is low enough so as to not interfere with a chosen minicell application or activity. A variety of buffers and media may be used during these purification procedures. These buffers are chosen for their ability to maintain the integrity of the minicells during the purification process. Buffers and media used in these procedures may serve as an osmo-protectant, stabilizing agent, or energy source, or may contain agents that limit the growth of contaminating parental cells.

Contaminating parental cells may be eliminated from minicell preparations by incubation under conditions that selectively kills dividing cells. Because minicells neither grow nor divide, they are resistant to such treatments. An example of conditions that prevent or kill dividing parental cells is treatment of a parent cell culture with an antibacterial agent, such as penicillin. Penicillin prevents cell wall formation and leads to lysis of dividing cells. Other agents may be used to prevent division of parental cells. Such agents include azide. Azide is a reversible inhibitor of electron transport, and thus prevents cell division. Additional examples of compounds capable of eliminating or inhibiting the division of parent cells include D-cycloserine and phage MS2 lysis protein. Khachatourians (U.S. Pat. No. 4,311,797) states that it may be desirable to incubate minicell/parent cell mixtures in brain heart infusion broth at 36° C. to 38° C. prior to the addition of penicillin G and further incubations.

Alternatively, lytic phage infection can be used to selectively kill, and preferably lyse, minicell producing parent cells. For example, although minicells can internally retain M13 phage in the plasmid stage of the M13 life cycle, they are refractory to infection and lysis by M13 phage. In contrast, minicell producing parent cells are infected and lysed by M13 and are thus are selectively removed from a mixture comprising parent cells and minicells. For example, a mixture comprising parent cells and minicells is treated with M13 phage at a multiplicity of infection (M.O.I.) of 5. The infection is allowed to continue to a point where ≧50% of the parent cells are lysed, preferably ≧75%, more preferably ≧95% most preferably ≧99%; and ≦25% of the minicells are lysed or killed, preferably ≦15%, most preferably ≦1%.

Another example of a method by which minicell producing parent cells can be selectively killed, and preferably lysed, exploits the presence of a conditionally lethal gene present in a chromosome of the parent cell. Induction of the chromosomal lethal gene results in the destruction of parent cells, but does not impact minicells as they lack the chromosome harboring the conditionally lethal gene. For example, a parent cell may contain a chromosomal integrated bacteriophage comprising a conditionally lethal gene, such the temperature sensitive repressor gene lambda cI857. Induction of this phage, which results in the destruction of the parent cells but not of the achromosomal minicells, is achieved by simply raising the temperature of the growth media. A preferred bacteriophage to be used in this method is one that kills or lyses the parent cells but does not produce infective particles. Expression of a toxic protein or proteins can also be used to selectively kill or lyse minicell producing parental cells. For example, expression of a phage holing gene can be used to lyse parental cells to improve the purity of minicell preparations.

Modified Forms of Gram-Negative Minicells

Gram-negative eubacterial cells and minicells are bounded by an inner membrane (IM), which is surrounded by a cell wall, wherein the cell wall is itself enclosed within an outer membrane (OM). In certain embodiments, it is desirable to use fully intact minicells to stimulate an immunogenic response. In different aspects of the invention, it is preferred to disrupt or degrade the outer membrane, cell wall or inner membrane of a eubacterial minicell. Such treatments can be used to increase or decrease the immunogenicity of a minicell.

Eubacterial cells and minicells with altered membranes and/or cell walls are called “POROPLASTS” “spheroplasts,” and “protoplasts.” Herein, the terms “spheroplast” and “protoplast” refer to spheroplasts and protoplasts prepared from minicells. In contrast, “cellular spheroplasts” and “cellular protoplasts” refer to spheroplasts and protoplasts prepared from cells. Also, as used herein, the term “minicell” encompasses not only minicellsper se but also encompasses POROPLASTS, spheroplasts and protoplasts.

In a poroplast, the eubacterial outer membrane and lipopolysaccharide components have been removed. In a spheroplast, portions of a disrupted eubacterial outer membrane or disrupted cell wall may remain associated with the inner membrane of the minicell. The membrane and cell wall of the spheroplast is nonetheless porous because the permeability of the disrupted outer membrane and cell wall has been increased. A membrane is “disrupted” when the membrane's structure has been treated with an agent or incubated under conditions that lead to the partial degradation of the membrane, thereby increasing the permeability thereof. In contrast, a membrane that has been “degraded” is essentially, for the applicable intents and purposes, removed. In preferred embodiments, irrespective of the condition of the outer membrane and cell wall, the eubacterial inner membrane is not disrupted. Additionally, membrane proteins displayed on the inner membrane are accessible to compounds that are brought into contact with the minicell, poroplast, spheroplast, protoplast or cellular poroplast.

Poroplasts

For various applications poroplasted minicells are capable of preserving the cytoplasmic integrity of the minicell while producing increased stability over that of naked protoplasts. Maintenance of the cell wall in poroplasted minicells increases the osmotic resistance, mechanical resistance and storage capacity over protoplasts while permitting passage of small and medium size proteins and molecules through the porous cell wall.

A poroplast is a Gram-negative bacterium that has its outer membrane removed. The production of poroplasts involves a modification of the procedure to make protoplasts to remove the outer membrane. Like protoplasts, measuring the total lipopolysaccharide that remains in the poroplast preparation may be used to monitor the removal of the outer membrane. Endotoxin kits and antibodies reactive against lipopolysaccharide may be used to measure lipopolysaccharide in solution; increasing amounts of soluble lipopolysaccharide indicates decreased retention of lipopolysaccharide by protoplasts, This assay thus makes it possible to quantify the percent removal of total outer membrane from the poroplasted minicells.

Several chemical and physical techniques have been employed to remove the outer membrane of Gram-negative bacteria. Chemical techniques include the use of EDTA in Tris to make cells susceptible to hydrophobic agents such as actinomycin. Lactic acid permeabilizes Gram-negative bacteria by disrupting the outer membrane. Physical techniques for removing the outer membrane include the use of osmodifferentiation to facilitate the disruption of the outer membrane.

Spheroplasts

A spheroplast is a bacterial minicell that has a disrupted cell wall or a disrupted outer membrane. Unlike eubacterial minicells and poroplasts that have a cell wall and can thus retain their shape despite changes in osmotic conditions, the absence of an intact cell wall in spheroplasts means that these minicells do not have a rigid form.

Protoplasts

A protoplast is a bacterium that has its outer membrane and cell wall removed. The production of protoplasts typically involves the use of lysozyme and high salt buffers to remove the outer membrane and cell wall. Various commercially available lysozymes can be used in such protocols. Measuring the total lipopolysaccharide that remains in the protoplast preparation is used to monitor the removal of the outer membrane. Commercially available endotoxin kits assays can be used to measure lipopolysaccharide in solution; increasing amounts of soluble lipopolysaccharide indicates decreased retention of lipopolysaccharide by protoplasts. This assay thus makes it possible to quantify the percent removal of total outer membrane from the minicells.

For minicell applications that utilize bacterial-derived minicells, it may be necessary to remove the outer membrane of Gram-negative cells and/or the cell wall of any bacterial-derived minicell. For Gram-positive bacterial cells, removal of the cell wall may be easily accomplished using lysozyme. This enzyme degrades the cell wall allowing easy removal of now soluble cell wall components from the pelletable protoplasted minicells. In a more complex system, the cell wall and outer membrane of Gram-negative cells may be removed by combination treatment with EDTA and lysozyme using a step-wise approach in the presence of an osmoprotecting agent. Examples of osmoprotectants include sucrose and glycerol.

It has been found that the concentration of the osmoprotectant sucrose, the cell wall digesting enzyme lysozyme, and chelator EDTA can be optimized to increase the quality of the protoplasts produced. Separation of either prepared Gram-negative spheroplasts prepared in either fashion from removed remaining lipopolysaccharide may occur through exposure of the spheroplast mixture to an anti-LPS antibody. The anti-LPS antibody may be covalently or non-covalently attached to magnetic, agarose, sepharose, sepheracyl, polyacrylamide, and/or sephadex beads. Following incubation, lipopolysaccharide is removed from the mixture using a magnet or slow centrifugation resulting in a protoplast-enriched supernatant.

Monitoring loss of LPS may occur through dot-blot analysis of protoplast mixtures or various commercially available endotoxin kit assays can be used to measure LPS in solution; increasing amounts of soluble LPS indicates decreased retention of LPS by protoplasts. This immunoassay may comprise a step of comparing the signal to a standard curve in order to quantify the percent removal of total outer membrane from the minicells. Lipopolysaccharide removal has also been measured by gas chromatography of fatty acid methyl esters.

Minicells from L-Form Eubacteria

L-form bacterial strains can be used to prepare antigenic minicells. L-form bacterial strains lack an outer membrane, a cell wall, a periplasmic space and extracellular proteases. Thus, in L-form Eubacteria, the cytoplasmic membrane is the only barrier between the cytoplasm and its surrounding environment.

Segregation of minicells from L-form eubacterial parent cells allows for the generation of minicells that are at least partially deficient in barriers that lie outside of the cytoplasmic membrane, thus providing direct access to components displayed on the minicell membrane. Thus, depending on the strains and methods of preparation used, minicells prepared from L-form eubacterial parent cells will be similar if not identical to various forms of poroplasts, spheroplasts and protoplasts. Displayed components that are accessible in L-form minicells include, but are not limited to, lipids, small molecules, proteins, sugars, nucleic acids and/or moieties that are covalently or non-covalently associated with the cytoplasmic membrane or any component thereof.

L-form Eubacteria that can be used in the methods of the invention include species of Escherichia, Streptomyces, Proteus, Bacillus, Clostridium, Pseudomonas, Yersinia, Salmonella, Enterococcus and Erwinia.

Assaying Minicells

Levels of minicell production can be evaluated using methods described herein. Relatively high levels of minicell production are generally preferred. Minicell production can be assessed by microscopic examination of late log-phase cultures. The ratio of minicells to normal cells and the frequency of cells actively producing minicells are parameters that increase with increasing minicell production.

Recombinant DNA Expression in Minicells

Recombinant expression of an antigen of interest typically requires the use of an expression element, such as an expression cassette or construct. The expression element contains an open reading frame encoding the antigen of interest that is introduced into an appropriate minicell producing parent cell to generate a minicell expression system. Expression elements of the invention may be introduced into a recipient eubacterial or eukaryotic minicell producing parent cell either as a DNA or RNA molecule, which may be a linear molecule or, more preferably, a closed covalent circular molecule. Expression from the expression element may occur through transient expression of the introduced sequence. Alternatively, permanent expression of the expression element may occur through the integration of the introduced expression cassette into the chromosome of the minicell producing parent cell.

A variety of recombinant expression systems can be used to produce the antigens for use with the disclosed invention. Any minicell producing parent cell that can be used to express an antigen of interest are suitable for use with the disclosed methods. Examples of recognized eubacterial hosts that may be used in the present invention include bacteria such as E. coli, Bacillus, Streptomyces, Pseudomonas, Salmonella, and Serratia.

Eubacterial expression systems utilize plasmid and viral expression vectors that contain replication sites and control sequences derived from a species compatible with the minicell producing parent cell line may be used. Suitable phage or bacteriophage vectors include λgt10 and λgt11. Suitable virus vectors may include pMAM-neo and pKRC. Appropriate eubacterial plasmid vectors include those capable of replication in E. coli, such as pBR322, pUC118, pUC119, ColE1, pSC101, and pACYC 184. Bacillus plasmids include pC194, pC221, and pT127. Suitable Streptomyces plasmids include p1J101 and Streptomyces bacteriophages such as C31. Pseudomonas plasmids are also known in the art.

To express an antigen in a eubacterial cell, typically one will operably link an open reading frame (ORF) encoding an antigen of interest to a functional promoter. Such promoters can be constitutive or more preferably, inducible. Examples of constitutive promoters include the int promoter of bacteriophage lambda, the bla promoter of the beta-lactamase gene sequence of pBR322, and the cat promoter of the chloramphenicol acetyl transferase gene sequence of pPR325. Examples of inducible eubacterial promoters include the major right and left promoters of bacteriophage lambda (P_{L} and P_{R}), the trp, recA, lacZ, lacI, and gal promoters of E. coli, the alpha-amylase and the sigma-28-specific promoters of B. subtilis, the promoters of the bacteriophages of Bacillus, and Streptomyces promoters.

Mammalian expression systems utilize host cells such as HeLa cells, cells of fibroblast origin such as VERO or CHO-K1, or cells of lymphoid origin and their derivatives. Preferred mammalian host cells include SP2/0 and J558L, as well as neuroblastoma cell lines such as IMR 332, which may provide better capacities for correct post-translational processing. Non-limiting examples of mammalian extrachromosomal expression vectors include pCR3.1 and pcDNA3.1, and derivatives thereof including those that are described by and are commercially available from INVITROGEN (Carlsbad, Calif.).

Several expression vectors are available for the expression of polypeptides in mammalian minicell producing parent cells. A wide variety of transcriptional and translational regulatory sequences may be employed, depending upon the nature of the minicell producing parent cell. The transcriptional and translational regulatory signals may be derived from viral sources, such as adenovirus, bovine papilloma virus, cytomegalovirus (CMV), simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals that are temperature-sensitive since, by varying the temperature, expression can be repressed or initiated, or are subject to chemical (such as metabolite) regulation.

Preferred eukaryotic plasmids include, for example, BPV, vaccinia, SV40, 2-micron circle, and the like, or their derivatives. Such plasmids are well known in the art (Botstein et al., Miami Wntr. Symp. 19:265-274, 1982; Broach, in: The Molecular Biology of the Yeast Saccharomyces: Life Cycle and Inheritance, Cold Spring Harbor Laboratory, Cold Spring Harbor, N.Y., p. 445-470, 1981; Broach, Cell 28:203-204, 1982; Bollon et al., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980).

Expression of polypeptides in eukaryotic hosts generally involves the use of eukaryotic regulatory regions. Such regions will, in general, include a promoter region sufficient to direct the initiation of RNA synthesis. Preferred eukaryotic promoters include, the promoter of the mouse metallothionein I gene, the TK promoter of Herpes virus, the SV40 early promoter, and the yeast gal4 gene sequence promoter.

Expression sequences and elements are also required for efficient expression. Examples of such sequences include Kozak and IRES elements in eukaryotes, and Shine-Delgarno sequences in prokaryotes, which direct the initiation of translation (Kozak, Initiation of translation in prokaryotes and eukaryotes. Gene, 1999. 234; 187-208; Martinez-Salas et al., Functional interactions in internal translation initiation directed by viral and cellular IRES elements, Jour. of Gen. Virol. 82:973-984, 2001); enhancer sequences; optional sites for repressor and inducers to bind; and recognition sites for enzymes that cleave DNA or RNA in a site-specific manner. Translation of mRNA is generally initiated at the codon, which encodes the first methionine residue; if so, it is preferable to ensure that the linkage between a eukaryotic promoter and a pre-selected open reading frame (ORF) does not contain any intervening codons that encode a methionine. The presence of such codons results either in the formation of a fusion protein with an uncharacterized N-terminal extension (if the AUG codon is in the same reading frame as the open reading frame) or a frame-shift mutation (if the AUG codon is not in the same reading frame as the open reading frame).

Expression of Antigenic Proteins

In a preferred embodiment, antigens of interest are expressed and presented on the surface of minicells. In one embodiment, antigens of interest are expressed as integral membrane proteins using a minicell producing expression system. The expressed antigens are displayed to a host immune system. Minicell producing cells or minicells harboring an expression vector are used to express the antigen of interest.

An “expression vector” is typically a nucleic acid encoding an open reading frame operably linked to one or more expression sequences that direct the expression of the open reading frame. The term “operably linked” means that the open reading frame is positioned with respect to expression sequences so that the amino acid sequence encoded by the open reading frame is faithfully transcribed, producing a gene product. The term “gene product” refers to either a nucleic acid (the product of transcription, reverse transcription, or replication) or a polypeptide (the product of translation) that is produced using the non-vector nucleic acid sequences as a template.

In one embodiment, it is preferable to use an expression construct that is an episomal expression construct. Minicells produced from a minicell producing cell line that has been transformed with an episomal expression construct will contain one or more of the expression constructs. These minicells are capable of expressing an open reading frame incorporated into the episomal expression construct. More specifically, these minicells will direct the production of the polypeptide encoded by the open reading frame using the RNA and ribosomal machinery that segregated into the minicell at the minicell budded off from the parent cell. At the same time, any mRNA molecules transcribed from a chromosomal gene prior to minicell formation that have been transferred to the minicell are degraded by endogenous RNases without being replaced by new transcription from the (absent) bacterial chromosome.

Chromosomal-encoded mRNAs will not be produced in minicells and will be “diluted” as increasing amounts of mRNAs transcribed from the episomal element are generated. A similar dilution effect is expected to increase the relative amount of episomally-generated proteins relative to any chromosome-encoded proteins present in the minicells. It is thus possible to generate minicells that are enriched for proteins encoded by and expressed from episomal expression constructs.

It is also possible to transform minicells with exogenous DNA after they have been prepared or separated from their parent cells. For example, phage RNA is produced in minicells after infection by lambda phage, even though replication of lambda phage may not occur in minicells.

Because it is the most characterized minicell-producing species, many of these episomal elements have been examined in minicells derived from E. coli. It is understood by practitioners of the art, however, that many episomal elements that are expressed in E. Coli also function in other eubacterial species, and that episomal expression elements for minicell systems in other species are available for use in the invention disclosed herein.

Eukaryotic and archaebacterial minicells can also be used for expression of membrane proteins. Use of eukaryotic and archaebacterial minicells may be desirable when an antigen of interest expressed in such a minicell has enhanced or altered activity after they undergo post-translational modification processes such as phosphorylation, proteolysis, mystrilation, GPI anchoring and glycosylation.

Expression elements comprising expression sequence operably linked to open reading frames encoding the membrane proteins of interest are transformed into eukaryotic cells according to methods and using expression vectors known in the art. By way of non-limiting example, primary cultures of rat cardiomyocytes have been used to produce exogenous proteins after transfection of expression elements therefor by electroporation.

Yeast cells that produce minicells are transformed with expression elements comprising an open reading frame encoding a membrane protein operably linked to yeast expression sequences. Cells that harbor a transferred expression element may be selected using a gene that is part of the expression element that confers resistant to an antibiotic, such as neomycin.

Alternatively, in one aspect of the invention, bacterial minicells are prepared that contain expression elements that are prepared from shuttle vectors. A “shuttle vector” has sequences required for its replication and maintenance in cells from two different species of organisms, as well as expression elements, at least one of which is functional in bacterial cells, and at least one of which is functional in yeast cells. For example, E. coli-yeast shuttle vectors are known in the art and include those derived from Yip, Yrp, Ycp and Yep. Preferred E. coli-yeast shuttle vectors are episomal elements that can segregrate into yeast minicells. Particularly preferred are expression vectors of the Yep (yeast episomal plasmid) class, and other derivatives of the naturally occurring yeast plasmid known as the 2 μm circle. The latter vectors have relatively high transformation frequencies and are stably maintained through mitosis and meiosis in high copy number. Expression of antigens in eubacterial systems comprising an inner and outer membrane can have the expressed antigenic protein directed to either the outer membrane, the periplasmic space, the inner membrane, or the cytoplasm.

Detecting Protein Synthesis in Minicells

Methods for detecting and assaying protein production are known in the art. For example, transformed E. coli minicell-producing cells are grown in LB broth with the appropriate antibiotic overnight. The following day the overnight cultures are diluted 1:50 in fresh media, and grown at 37° C. to mid-log phase. If it is desired to eliminate whole cells, an antibiotic that kills growing (whole) cells but not quiescent cells (minicells) may be used. For example, in the case of cells that are not ampicillin resistant, ampicillin (100 mg per ml is added), and incubation is allowed to continue for about 2 more hours. Cultures are then centrifuged twice at low speed to pellet most of the large cells. Minicells are pelleted by spinning 10 minutes at 10,000 rpm, and are then resuspended in M63 minimal media supplemented with 0.5% casamino acids, and 0.5 mM cAMP, or M9 minimal medium supplemented with 1 mM MgSO₄, 0.1 mM CaCl₂, 0.05% NaCl 0.2% glucose, and 1 ng per ml thiamine. Labeled (³⁵S) methionine is added to the minicells for about 15 to about 90 minutes, and minicells are immediately collected afterwards by centrifugation for 10 min at 4° C. and 14,000 rpm. Cells are resuspended in 50 to 100 μg Laemmeli-buffer, and disrupted by boiling and vortexing (2 minutes for each step). Incorporation of ³⁵S-methionine was determined by measuring the amount of radioactivity contained in 141 of the lysate after precipitation of proteins with trichloroacetic acid (TCA). Minicell lysates (50,000 to 100,000 cpm per lane) are subjected to 10% PAGE. Gels are fixed and images there of are generated by autoradiography or any other suitable detection systems.

Minicell Modifications

A variety of compounds or moieties can be chemically attached (conjugated) to minicells via membrane proteins that are displayed on the minicells. The compound to be conjugated to minicells (the “attachable compound”) may of any chemical composition, for example, small molecules, nucleic acids, radioisotopes, lipids or polypeptides.

It is possible to prepare minicells that express transmembrane proteins with cysteine moieties on extracellular domains. Linkage of the membrane protein may be achieved through surface cysteinyl groups by, for example, reduction with cysteinyl residues on other compounds to form disulfide bridges. If appropriate cysteinyl residues are not present on the membrane protein they may be introduced by genetic manipulation. To illustrate, bioactive lysosphingolipids (such as sphingosine, sphingosine-1-phosphate, and sphingosylphosphoryl choline) can be covalently linked to proteins expressed on the surfaces of minicells such that these bioactive lipids are on the surface of the minicells.

When the attachable moiety and the membrane protein both have a reduced sulfhydryl group, a homobifunctional cross-linker that contains maleimide, pyridyl disulfide, or beta-alpha-haloacetyl groups may be used for cross-linking. Examples of such cross-linking reagents include bismaleimidohexane (BMH⁺) or 1,4-Di-[3′-(2′-pyridyldithio)propionamido]butane (DPDPB). Alternatively, a heterobifunctional cross-linker that contains a combination of maleimide, pyridyl disulfide, or beta-alpha-haloacetyl groups can be used for cross-linking.

Attachable moieties may also be chemically conjugated using primary amines. In these instances, a homobifunctional cross-linker that contains succiminide ester, imidoester, acylazide, or isocyanate groups may be used for cross-linking. Examples of such cross-linking reagents include, but are not limited to: Bis[2-(succinimidooxycarbonyloxy)ethyl]sulfone (BSOCOES); Bis[2-(sulfosuccinimidooxycarbonyloxy)ethyl]sulfone (sulfo-BSCOCOES); Disuccinimidyl suberate (DSS); Bis-(Sulfosuccinimidyl) Suberate (BS3); Disuccinimidyl glutarate (DSG); Dithiobis(succinimidylpropionate) (DSP); Dithiobois(sulfosuccinimidylpropionate) (DTSSP); Disulfosuccinimidyl tartrate (sulfo-DST); Dithio-bis-maleimidoethane (DTME); Disuccinimidyl tartrate (DST); Ethylene glycolbis(sulfosuccinimidylsuccinate) (sulfo-EGS); Dimethyl malonimidate•2 HCl (DMM); Ethylene glycolbis(succinimidylsuccinate) (EGS); Dimethyl succinimidate•2 HCl (DMSC); Dimethyl adipimidate•2 HCl (DMA); Dimethyl pimelimidate•2 HCl (DMP); and Dimethyl suberimidate•2-HCl (DMS), and Dimethyl 3,3′-dithiobispropionimidate•2 HCl (DTBP). Heterobifunctional cross-linkers that contains a combination of imidoester or succinimide ester groups may also be used for cross-linking.

Attachable moieties may also be chemically conjugated using sulfhydryl and primary amine groups. In these instances, heterobifunctional cross-linking reagents are preferable used. Examples of such cross-linking reagents include, but are not limited to: N-succinimidyl 3-(2-pyridyldithio)propionate (DPDP); N-succinimidyl 6-[3′-(2-pyridyldithio)-propionamido] hexanoate (sulfo-LC-SPDP); m-maleimidobenzoyl-N-hydroxysuccinimide ester (MBS); m-maleimidobenzoyl-N-hydroxysulfosuccinimide ester (sulfo-MBS); succinimidyl 4-[P-maleimidophenyl] butyrate (SMPB); sulfosuccinimidyl 4-[p-maleimidophenyl] butyrate (sulfo-SMPB); N-[4-Maleimidobutyryloxy] succinimide ester (GMBS), N-[4-Maleimidobutyryloxy]sulfosuccinimide ester (sulfo-GMBS); N-[4-maleimidocaproyloxy] succinimide ester (EMCS); N-[4-maleimidocaproyloxy]sulfosuccinimide ester (sulfo-EMCS); N-succinimidyl(4-iodoacetyl)aminobenzoate (SIAB); sulfosucinimidyl(4-iodacetyl)aminobenzoate (sulfo-SIAB); succinimidyl 4-(N-maleimidomethyl)cyclohexane-1-carboxylate (SMCC); sulfosuccinimidyl 4-(N-maleimidomethyl)cyclohexane-1-carboxylate (sulfo-SMCC); succiminidyl-4-(N-maleimidomethyl)cyclohexane-1-carboxy-(6-amido-caproate) (LC-SMCC); 4-succinimidyloxycarbonyl-methyl-(2-pyridyldithio) toluene (SMPT); and sulfo-LC-SMPT.

As an exemplary protocol, a minicell suspension is made 5 mM EDTA/PBS, and a reducing solution of 2-mercaptoethylamine in 5 mM EDTA/PBS is added to the minicells. The mixture is incubated for 90 minutes at 37° C. The minicells are washed with EDTA/PBS to remove excess 2-mercaptoethylamine. The attachable moiety is dissolved in PBS, pH 7.2. A maleimide crosslinker is added to the solution, which is then incubated for 1 hour at room temperature. Excess maleimide is removed by column chromatography.

The minicells with reduced sulfhydryl groups are mixed with the derivatized compounds having an attachable moiety. The mixture is allowed to incubate at 4° C. for 2 hours or overnight to allow maximum coupling. The conjugated minicells are washed to remove unreacted (unattached) compounds having the attachable moiety. Similar protocols are used for expressed membrane proteins with other reactive groups (e.g., carboxyl, amine) that can be conjugated to an attachable moiety.

Formulation and Administration of Immunogenic Minicells

Formulations of immunogenic minicells include a suitable carrier. Because minicells may be destroyed by digestion or prevented from acting due to antibody secretion in the gut, they are preferably administered parenterally, including, for example, administration that is subcutaneous, intramuscular, intravenous, intradermal, nasal, mucosal, or via suppository. Formulations suitable for parenteral administration include aqueous and non-aqueous sterile injection solutions that may contain buffers, and solutes which render the formulation isotonic with the bodily fluid, preferably the blood, of the individual; and aqueous and non-aqueous sterile suspensions which may include suspending agents or thickening agents. The formulations may be presented in unit-dose or multi-dose containers, for example, sealed ampules and vials and may be stored in a freeze-dried condition requiring only the addition of the sterile liquid carrier immediately prior to use.

The immunogenic formulations disclosed herein may include adjuvant systems for enhancing the immunogenicity of the formulation. Adjuvants are substances that can be used to augment or modulate an immune response. Typically an adjuvant and an antigen of interest are mixed prior to presentation to the immune system. Alternatively, the adjuvant and the antigen are presented separately. Examples of materials suitable for use in vaccine compositions are provided in Osol, A., ed., Remington's Pharmaceutical Sciences, Mack Publishing Co, Easton, Pa. (1980), pp. 1324-1341, which reference is entirely incorporated herein by reference.

Compositions comprising immunogenic minicells are injected into a human or animal at a dosage of about 0.1-1000 μg per kg body weight. Antibody titers against antigens of interest are determined by ELISA, using the recombinant protein and horseradish peroxidase-conjugated goat anti-human or animal immunoglobulins or other serologic techniques. Cellular immune responses to immunogenic minicells can also be measured using various assays well known to those of ordinary skill in the art. Booster injections are administered as needed to achieve the desired levels of protective antibodies or T cells.

Routes and frequency of administration, as well as the dosage of immunogenic minicell preparations will vary from individual to individual. Between 1 and 10 doses may be administered for a 52-week period. Preferably, 6 doses are administered, at intervals of 1 month, and booster administrations may be given periodically thereafter. Alternate protocols may be appropriate for individual patients.

Immunotherapy of hyperproliferative disorders using immunogenic minicell preparations typically comprises providing a suitable dose of minicells to a subject in need thereof. Efficacy of such a treatment can be monitored, as described above, by determining the degree an anti-tumor immune response results in response to the administration of the immunogenic minicells. The immune response of an immunized subject can be monitored by measuring the anti-tumor antibodies in a patient or by immunogen-dependent generation of cytolytic effector cells capable of killing the patient's tumor cells in vitro. Typically, an effective dose of a particular immunogenic minicell composition is capable of causing an immune response that leads to an improved clinical outcome in immunized subjects as compared to non-immunized subjects.

The immunogenic compositions according to the invention may contain a single or multiple species of immunogenic minicells where each species displays a different immunogen. Additionally or alternatively, immunogenic minicells may each display or express one or more immunogen.

### Pharmaceutical Compositions

Another aspect of the invention is drawn to compositions, including pharmaceutical compositions. A “composition” refers to a mixture comprising at least one carrier, preferably a physiologically acceptable carrier, and one or more immunogenic minicell compositions. The term “carrier” defines a chemical compound that does not inhibit or prevent the incorporation of the immunologically active peptide(s) into cells or tissues. A carrier typically is an inert substance that allows an active ingredient to be formulated or compounded into a suitable form. Exemplary forms include a pill, a capsule, a gel, a film, a tablet, a microparticle, a solution, an ointment, a paste, an aerosol, a droplet, a colloid or an emulsion etc.

A “physiologically acceptable carrier” is a carrier suitable for use under physiological conditions that does not abrogate (reduce, inhibit, or prevent) the immunological activity and properties of the compound. For example, dimethyl sulfoxide (DMSO) is a carrier as it facilitates the uptake of many organic compounds into the cells or tissues of an organism. Preferably, the carrier is a physiologically acceptable carrier, preferably a pharmaceutically acceptable carrier, in which the immunogenic minicell composition is disposed.

A “pharmaceutical composition” refers to a composition wherein the carrier is suitable for use in humans or other animals. The term “pharmaceutically acceptable carrier” includes any medium or material that is not biologically or otherwise undesirable. The carrier may be administered to an organism along with an immunogenic minicell composition without causing undesirable effects or interacting in a deleterious manner with the complex or any of its components or the organism. Examples of pharmaceutically acceptable reagents are provided in The United States Pharmacopeia, The National Formulary, United States Pharmacopeial Convention, Inc., Rockville, Md. 1990, hereby incorporated by reference herein into the present application.

The terms “therapeutically effective amount” or “pharmaceutically effective amount” mean an amount sufficient to induce or effectuate a measurable immunogenic response in the target cell, tissue, or body of an organism. What constitutes a therapeutically effective amount will depend on a variety of factors, which the knowledgeable practitioner will take into account in arriving at the desired dosage regimen.

The compositions of the invention can further comprise other chemical components, such as diluents and excipients. A “diluent” is a chemical compound diluted in a solvent, preferably an aqueous solvent, that facilitates dissolution of the composition in the solvent, and it may also serve to stabilize the immunogenic composition or one or more of its components. Salts dissolved in buffered solutions are utilized as diluents in the art. For example, preferred diluents are buffered solutions containing one or more different salts. A preferred buffered solution is phosphate buffered saline (particularly in conjunction with compositions intended for pharmaceutical administration), as it mimics the salt conditions of human blood. Since buffer salts can control the pH of a solution at low concentrations, a buffered diluent rarely modifies the activity of an immunologically active peptide.

An “excipient” is any more or less inert substance that can be added to a composition to confer a suitable property thereto. Suitable excipients and carriers include, in particular, fillers such as sugars, including lactose, sucrose, mannitol, or sorbitol cellulose preparations such as, for example, maize starch, wheat starch, rice starch, agar, pectin, xanthan gum, guar gum, locust bean gum, hyaluronic acid, casein potato starch, gelatin, gum tragacanth, methyl cellulose, hydroxypropylmethyl-cellulose, polyacrylate, sodium carboxymethylcellulose, and/or polyvinylpyrrolidone (PVP). If desired, disintegrating agents can also be included, such as cross-linked polyvinyl pyrrolidone, agar, or alginic acid or a salt thereof such as sodium alginate. Other suitable excipients and carriers include hydrogels, gellable hydrocolloids, and chitosan. Chitosan microspheres and microcapsules can be used as carriers. See WO 98/52547 (which describes microsphere formulations for targeting compounds to the stomach, the formulations comprising an inner core (optionally including a gelled hydrocolloid) containing one or more active ingredients, a membrane comprised of a water insoluble polymer, such as ethylcellulose, to control the release rate of the active ingredient(s), and an outer layer comprised of a bioadhesive cationic polymer, for example, a cationic polysaccharide, a cationic protein, and/or a synthetic cationic polymer; U.S. Pat. No. 4,895,724. Typically, chitosan is cross-linked using a suitable agent, for example, glutaraldehyde, glyoxal, epichlorohydrin, and succinaldehyde. Compositions employing chitosan as a carrier can be formulated into a variety of dosage forms, including pills, tablets, microparticles, and microspheres, including those providing for controlled release of the active ingredient(s). Other suitable bioadhesive cationic polymers include acidic gelatin, polygalactosamine, polyamino acids such as polylysine, polyhistidine, polyomithine, polyquaternary compounds, prolamine, polyimine, diethylaminoethyldextran (DEAE), DEAE-imine, DEAE-methacrylate, DEAE-acrylamide, DEAE-dextran, DEAE-cellulose, poly-p-aminostyrene, polyoxethane, copolymethacrylates, polyamidoamines, cationic starches, polyvinylpyridine, and polythiodiethylaminomethylethylene.

The immunogenic minicell compositions of the invention can be formulated in any manner suitable for administration. Immunogenic minicell compositions may be uniformly (homogeneously) or non-uniformly (heterogeneously) dispersed in the carrier. Suitable formulations include dry and liquid formulations. Dry formulations include freeze dried and lyophilized powders, which are particularly well suited for aerosol delivery to the sinuses or lung, or for long term storage followed by reconstitution in a suitable diluent prior to administration. Other preferred dry formulations include those wherein a composition according to the invention is compressed into tablet or pill form suitable for oral administration or compounded into a sustained release formulation.

When the composition is intended for oral administration but is to be delivered to epithelium in the intestines, it is preferred that the formulation be encapsulated with an enteric coating to protect the formulation and prevent premature release of the immunogenic minicell compositions included therein. As those in the art will appreciate, the compositions of the invention can be placed into any suitable dosage form. Pills and tablets represent some of such dosage forms.

The compositions can also be encapsulated into any suitable capsule or other coating material, for example, by compression, dipping, pan coating, spray drying, etc. Suitable capsules include those made from gelatin and starch. In turn, such capsules can be coated with one or more additional materials, for example, and enteric coating, if desired. Liquid formulations include aqueous formulations, gels, and emulsions.

In certain preferred embodiments the immunogenic compositions that comprise a bioadhesive, preferably a mucoadhesive, coating. A “bioadhesive coating” is a coating that allows a substance (e.g., a minicell composition) to adhere to a biological surface or substance better than occurs absent the coating. A “mucoadhesive coating” is a preferred bioadhesive coating that allows a substance, for example, a composition according to the invention, to adhere better to mucosa occurs absent the coating. For example, micronized particles having a mean diameter of about 5, 10, 25, 50, or 100 μm can be coated with a mucoadhesive. The coated particles can then be assembled into a dosage form suitable for delivery to an organism. Preferably, and depending upon the location where the cell surface transport moiety to be targeted is expressed, the dosage form is then coated with another coating to protect the formulation until it reaches the desired location, where the mucoadhesive enables the formulation to be retained while the composition interacts with the target cell surface transport moiety.

The immunogenic minicell compositions of the invention may be administered to any organism, preferably an animal, preferably a mammal, bird, fish, insect, or arachnid. Preferred mammals include bovine, canine, equine, feline, ovine, and porcine animals, and non-human primates. Humans are particularly preferred. Multiple techniques of administering or delivering a compound exist in the art including, but not limited to, oral, rectal (enema or suppository), aerosol (nasal or pulmonary delivery), parenteral, and topical administration.

Preferably, sufficient quantities of the immunogenic minicell composition are delivered to achieve the intended effect. The particular amount of composition to be delivered will depend on many factors, including the effect to be achieved, the type of organism to which the composition is delivered, delivery route, dosage regimen, and the age, health, and sex of the organism. As such, the particular dosage of a composition incorporated into a given formulation is left to the ordinarily skilled artisan's discretion.

Those skilled in the art will appreciate that when the immunogenic minicell compositions of the invention are administered as agents to achieve a particular desired immunological result. The desired immunological result may include a therapeutic or protective effect. Suitable formulations and methods of administration of therapeutic agents include those for oral, pulmonary, nasal, buccal, ocular, dermal, rectal, or vaginal delivery.

Depending on the mode of delivery employed, the context-dependent functional entity can be delivered in a variety of pharmaceutically acceptable forms. For example, the context-dependent functional entity can be delivered in the form of a solid, solution, emulsion, dispersion, micelle, liposome, and the like, incorporated into a pill, capsule, tablet, suppository, aerosol, droplet, or spray. Pills, tablets, suppositories, aerosols, powders, droplets, and sprays may have complex, multilayer structures and have a large range of sizes. Aerosols, powders, droplets, and sprays may range from small (1 micron) to large (200 micron) in size.

Pharmaceutical compositions of the present invention can be used in the form of a solid, a lyophilized powder, a solution, an emulsion, a dispersion, a micelle, a liposome, and the like, wherein the resulting composition contains one or more of the compounds of the present invention, as an active ingredient, in admixture with an organic or inorganic carrier or excipient suitable for enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. Examples of a stabilizing dry agent includes triulose, preferably at concentrations of 0.1% or greater (See, e.g., U.S. Pat. No. 5,314,695, which is hereby incorporated in its entirety). The active compound is included in the pharmaceutical composition in an amount sufficient to produce the desired effect upon the process or condition of diseases.

Antigens From Category A Pathogens

Any antigen, including pathogenic and cancerous antigens, can be used with the immunogenic minicells described herein. For example, pathogens from the NIAID Category A pathogen list can be used herein, and are discussed in detail below. A brief description of the NIAID Category A pathogen is provided along with a list of potential antigenic targets and accession numbers encoding those targets.

Bacillus anthracis (Anthrax)

Anthrax is caused by B. anthracis. This organism is an extracellular toxinogenic bacterium. An anthrax vaccine comprising minicells that express spore antigens as well as antigens from the anthrax toxins is disclosed.

Spore Surface

The exosporium is the most external structure of the spore form of B. anthracis. A glycoprotein termed BclA (for Bacillus collagen-like) constituent of the exosporium has been identified. It contains a central region presenting similarity to mammalian collagen proteins. BclA is the structural component of the filaments located at the surface of the exosporium.

Toxins of Bacillus anthracis

Bacillus anthracis secretes two toxins composed of three proteins. The first toxin is the lethal toxin, which is composes of a protective antigen (PA) and a lethal factor (LF). The second toxin is the edema toxin, which is composed a PA and an edema factor (EF). The PA (protective antigen) is the common component able to bind and deliver EF (edema factor) and LF (lethal factor) into target eukaryotic cells. EF is a calmodulin-dependent adenylate cyclase and LF is a metalloprotease.

Forms of Bacillus anthracis

The vegetative form of the bacilli is encapsulated. The capsule covers a structural array termed S-layer. The S-layer is composed of two abundant proteins, Sap and EA1. The capsule is composed of at least three proteins, which genes belong to an operon, are necessary for capsule synthesis. There is a fourth protein encoded by the same operon that regulates capsule levels by degrading excess capsule protein. Provided below is a table of various genes of Bacillus anthracis that can be expressed by minicells to generate immunogenic compositions.

| Bacillus | | | Accession |
|---|---|---|---|
| anthracis | Protein | Strain | Numbers |
| | Bc1A genes | ATCC4229 | AJ516947 |
| | | CIP5725 | AJ516946 |
| | | RA3 | AJ516945 |
| | | 7611 | AJ516944 |
| | | ATCC6602 | AJ516943 |
| | | 6183 | AJ516942 |
| | | 9602 | AJ516941 |
| | | CIPA2 | AJ516940 |
| | | CIP53169 | AJ516939 |
| | | CIP8189 | AJ516938 |
| | | CIP7702 | AJ516937 |
| | | Ames | AJ516936 |
| | PA genes | virulence plasmid pX01 | NC_001496 |
| | | A2012 plasmid pXO1 | NC_003980 |
| | | BA1024 | AF306783 |
| | | plasmid pX01 | AF306782 |
| | | isolate 33 | AF306781 |
| | | isolate BA1035 | AF306780 |
| | | isolate 28 | AF306779 |
| | EF genes | virulence plasmid pX01 | NC_001496 |
| | | isolate IT-Carb3-6249 | AJ413931 |
| | | isolate IT-Carb1-6225 | AJ413930 |
| | | isolate Sterne | M24074 |
| | | isolate Sterne | M23179 |
| | LF genes | virulence plasmid pX01 | NC_001496 |
| | | A2012 plasmid pXO1 | NC_003980 |
| | | clone: pLF74 | M29081 |
| | | virulence plasmid pX01 | M30210 |
| | SAP genes | isolate Sterne | Z36946 |
| | EA1 genes | isolate Sterne | X99724 |
| | Capsule genes | A2012 plasmid pXO2, | NC_003981 |
| | | CapA, B, & C | |
| | | plasmid pX02 capR | AB017611 |

Clostridium botulinum (Botulism)

Clostridium botulinum is an anaerobic, rod-shaped spore producing bacterium that produces a protein with characteristic neurotoxicity. Antigenic types of C. botulinum are identified by complete neutralization of their toxins by the homologous antitoxin; cross-neutralization by heterologous antitoxins does not occur or is minimal. There are seven recognized antigenic types: A, B, C, D, E, F, and G. Types C and D are not thought to cause human disease, however this has not been definitively established.

Cultures of five of these types apparently produce only one type of toxin but all are given type designations corresponding to their toxin production. Types C and D cross-react with antitoxins to each other because they each produce more than one toxin and have at least one common toxin component. Type C produces predominantly C₁ toxin with lesser amounts of D and C₂, or only C₂, and type D produces predominantly type D toxin along with smaller amounts of C₁ and C₂. Mixed toxin production by a single strain of C. botulinum may be more common than previously realized. There is a slight reciprocal cross-neutralization with types E and F, and recently a strain of C. botulinum was shown to produce a mixture of predominantly type A toxin, with a small amount of type F.

C. botulinum is widely distributed in soils and in sediments of oceans and lakes. The finding of type E in aquatic environments by many investigators correlates with cases of type E botulism that were traced to contaminated fish or other seafood. Types A and B are most commonly encountered in foods subjected to soil contamination. In the United States, home-canned vegetables are most commonly contaminated with types A and B, but in Europe, meat products have also been important vehicles of foodborne illness caused by these types.

Provided below is a table of various genes of C. botulinum that can be expressed by minicells to generate immunogenic compositions.

| | | | Accession |
|---|---|---|---|
| Clostridium botulinum | Protein | Strain | Numbers |
| | Neurotoxin Type A | 5′ end | M27892 |
| | | isolate Kumgo, light chain- | AY166872 |
| | | partial cds | |
| | | Synthetic construct | AF464912 |
| | Neurotoxin Type B | Isolate 1436 | AF295926 |
| | | isolate 13280 | AF300469 |
| | | isolate 667 | AF300468 |
| | | isolate 519 | AF300467 |
| | | isolate 593 | AF300466 |
| | | isolate 588 | AF300465 |
| | Neurotoxin Type C | Isolate 6813 | D49440 |
| | | Botulinum bacteriophage | X62389 |
| | | Bacteriophage c-st | D90210 |
| | | C2 toxin component-I and | D88982 |
| | | component-II | |
| | | C2 toxin (component-I) | D63903 |
| | Neurotoxin Type D | BVD/−3 | X54254 |
| | Neurotoxin Type E | Isolate 35396 | AB082519 |
| | | Hazen 36208 | X70815 |
| | | VH Dolman | X70818 |
| | | NCTC 11219 | X62683 |
| | | Beluga | X62089 |
| | Neurotoxin Type F | 202F | Y10770 |
| | | proteolytic F Langeland | X70821 |
| | | non-proteolytic Hobbs FT10 | X70820 |
| | | non-proteolytic Craig 610 | X70816 |
| | | 202F | M92906 |
| | Neurotoxin Type G | synthetic sequence based on | AX608812 |
| | | Wild type | |
| | | 113/30, NCFB 3012 | X74162 |

Yersinia pestis

Yersinia pestis is the causative agent of plague. The nucleotide sequence of the organism's genome has been elucidated. “Genome sequence of Yersinia pestis, the causative agent of plague,” Nature 413 523-527.

Y. pestis has been extensively studied and this work provides a number of potential targets for a subunit vaccine. Prime candidates include the F1 antigen (cfa1), pla, the V antigen (LcrV), and Yops. The YscC protein has been advanced identified as residing in the outer member of the organism and as such could also provide a potential vaccine target. See Clin Microbiol Rev. 10(1):35-66 (1997) for a more complete review.

Provided below is a table of various genes of Y. pestis that can be expressed by minicells to generate immunogenic compositions.

| Yersinia | | | Accession |
|---|---|---|---|
| pestis | Protein | Strain | Numbers |
| | Complete Genome | CO92 | NC_003143 |
| | | KIM | NC_004088 |
| | F1 Antigen (caf1) | caf1, caf1M, caf1A and | X61996 |
| | | caf1R | |
| | pla gene | KIM | AF053945 |
| | V antigen | CO-92 Biovar Orientalis | NC_003131 |
| | | Angola | AF167310 |
| | | Pestoides | AF167309 |
| | Yops | CO-92 Biovar Orientalis | NC_003131 |
| | | KIM5 | AF074612 |
| | | KIM | AF053946 |

Variola Major (smallpox) and Other Pox Viruses

The smallpox virus genome has been completely sequenced.

| | | | Accession |
|---|---|---|---|
| Organism | Protein | Strain | Numbers |
| Variola major | Genome | India-1967, ssp. | NC_001611 |
| (smallpox) | sequence | major | |
| | | strain Bangladesh-1975 | L22579 |
| Vaccinia Virus | Genome | Ankara | U94848 |
| | Sequence | | |
| | Other | genomic DNA, 42 kbp | D11079 |
| | Sequences | | |
| | | Various genes | D00382 |
| | | Various genes | M36339 |
| | | Various genes | AF411106 |
| | | Various genes | AF411105 |
| | | Various genes | AF411104 |
| | | a13L ortholog gene | AJ309902 |
| | | for p8 | AJ315004 |
| | | A36R gene for p43-50 | AF120160 |
| | | protein | |
| | | DNA glycosylase (D4R | L24385 |
| | | and D5R) genes | |
| | | Various genes | M57977 |
| | | A33R (A33R) gene | AF226618 |
| | | L1R (L1R) gene | AF226617 |
| | | serine proteinase | D00582 |
| | | inhibitor | |

Francisella tularensis (Tularemia)

Francisella tularensis is a small gram-negative coccobacillus. There are two main serotypes: Jellison Types A and B. Type A is considered the more virulent form. F. tularensis may be aerosolized in dry or wet form.

### Viral Hemorrhagic Fevers

### Arenaviruses

Arenavirues are enveloped. The surface of the virion envelope is studded with glycoprotein projections that consist of tetrameric complexes of the viral glycoproteins GP1 and GP2. Obtaining gene and amino acid sequences for these proteins from each of the arenaviruses listed below would be helpful in supporting a vaccine patent application.

### LCM

Lymphocytic chorio meningitis (LCM) is caused by the lymphocytic chorio meningitis virus (LCMV). Studies in mice have shown that passive immunity is effective in protecting suckling mice from LCMV challenge. Because a protective immune response has been demonstrated, it may be possible to use minicell technology to produce large amounts of antibodies to treat victims of LCMV. Of course, a vaccine effective against LCMV would be a primary goal. LCMV appears to interact with CD+8 cells as part of its life cycle. The viral protein or proteins involved in this interaction would make prime targets for vaccine antigens using the disclosed minicell technology.

| Organism | Protein | Strain | Accession Numbers |
|---|---|---|---|
| LCMV | GP1 | WE | AJ233161 |
| | | Docile | AJ249159 |
| | | Docile | AJ249158 |
| | GP-C | CHV2 | U10158 |
| | | CHV3 | U10159 |
| | | CHV1 | U10157 |

### Junin Virus

The Junin virus is a member of Arenaviridae. It is pleomorphic, enveloped globular virions 110-130 nm in diameter, linear, single-stranded, two-segmented RNA. Junin virus found mainly in Argentina and causes Argentinian hemorrhagic fever. Potenital antigenic targets from the Junin virus include the GP1 and GP2 envelope glycoproteins. The NP, L and Z proteins are thought to be internal to the viral particle and would seem to be less likely targets. Nevertheless, these proteins can also be used to generate immunogenic compositions.

| Organism | Protein | Strain | Accession Numbers |
|---|---|---|---|
| Junin Virus | G1 (Partial) | PH3190 | AF264235 |
| | | PH7994 | AF264234 |
| | | PAn14823 | AF264233 |
| | | PH2412 | AF264232 |
| | GP-C | MC2 | D10072 |
| | NP | Vaccine Strain | U70804 |
| | Segment L | N/A | N/A |
| | Z | N/A | N/A |

Other arenavirues of interest include the Machupo virus, the Guanarito virus and the Lassa Fever genomic information and accession numbers for antigens of interest are provided below.

| Organism | Protein | Strain | Accession Numbers |
|---|---|---|---|
| Machupo | S Segment | Carvallo | AY129248 |
| Viruso | (Glycoprotein) | | |
| | | Carvallo | AF485260 |
| | nucleocapsid protein | AA288-77 | X62616 |
| Gaunarito | S Segment | INH-95551 | AY129247 |
| | (Glycoprotein) | | |
| | | INH-95551 | AF485258 |
| | nucleocapsid protein | VHF-5603 | AF204207 |
| | (Partial) | | |
| | | VHF-1150 | AF204206 |
| | | S-16995 | AF204205 |
| | | VHF-3990 | AF204204 |
| Lassa Virus | GP1 | 803213 | AF181854 |
| | | LP | AF181853 |
| | | 11620 | M15076 |
| | Segment L | Josiah | NC_004297 |
| | Segment S | Josiah | NC_004296 |
| | GP-C | AV | AF246121 |
| | Nucleoprotein | 11620 | J04324 |
| | | 803213 | AF181854 |
| | | LP | AF181853 |

### Bunyaviruses:

Bunyaviruses are spherical particles that display surface glycoprotein projections of 5 to 10 nm, which are embedded in a lipid bilayered envelope approximately 5 to 7 nm thick. Depending on the virus, there can be from 270 to 1,400 glycoprotein spikes per virion. The spikes are generally thought to consist of heterodimers of the viral glycoproteins G1 and G2. The G1 and G2 proteins are decorated with N-linked carbohydrates, so this may make these viruses less attractive as targets. Two bunyavitrses of interest are the hantavirus and the Rift Valley fever virus. Sequence information for these viruses is provided below.

| | | | Accession |
|---|---|---|---|
| Organism | Protein | Strain | Numbers |
| Hantavirus | G1 & G2 | cl-1 | D25529 |
| | | 84FLi | AF366569 |
| | | B-1 | X53861 |
| | M Polypeptide | 84FLi | AF345636 |
| | | MF-43 | AJ011648 |
| | | 84FLi | AF366569 |
| | | Ls136V | AJ011647 |
| Rift Valley Fever | Complete genome | ZH-548M12 | NC_002044 |
| Virus | | | |
| | G1 & G2 | Rift Valley Fever | M11157 |
| | | Virus | |

### Flaviviruses

There are a number of members of flavividae that are particularly attractive to serve as targets for a minicell immunogenic composition. Examples of pathogenic flaviviruses include the hepatitis C virus, St. Louis encephalitis virus, dengue, and a number of other encephalitis viruses. Sequence information for these viruses is provided below.

| | | | Accession |
|---|---|---|---|
| Organism | Protein | Strain | Numbers |
| Hepatitis C | Complete genome | M1LE | AB080299 |
| | | H77 | NC_004102 |
| | E protein | JB2-8 | AJ511254 |
| | | JB28-1 | AJ511253 |
| | | JB15-10 | AJ511252 |
| | M protein | N/A | N/A |
| | C protein | Gabonese | S73403 |
| | | Gabonese | S73404 |
| | | Gabonese | S73421 |
| St Louis encephalitis | E protein (partial) | BFS508 | AF112392 |
| | | FL79-411 | AF112391 |
| | | Hubbard | AF112389 |
| | M protein | N/A | N/A |
| | C protein | N/A | N/A |
| Dengue | Complete genome | 814669 | AF326573 |
| | | rDEN4del30 | AF326827 |
| | | 2Adel30 | AF326826 |
| | | N/A | NC_001474 |

### Filoviruses

Filoviruses are enveloped viruses that can cause hemorrhagic fevers. Exemplary filoviruses include the Ebola and Marburg viruses. There are four species of Ebola-like viruses: Zaire, Sudan, Reston, and Côte d'Ivoire. There is only one representative of the Marburg filovirus. Each of these viruses contains VP40, GP, and VP24 proteins that are thought to be membrane-associated proteins. These proteins are candidates for vaccine targets using the disclosed minicell technology.

| | | | Accession |
|---|---|---|---|
| Organism | Protein | Strain | Numbers |
| Zaire Ebola virus | Complete genome | Mayinga | NC_002549 |
| | | Mayinga | AY142960 |
| Sudan Ebola virus | NP | Boniface | AF173836 |
| | SP | Boniface | U28134 |
| | L | Maleo 1979 | U23458 |
| | GP | Maleo | U23069 |
| Reston Ebola virus | Complete genome | Pennsylvania | NC_004161 |
| | | Reston | AB050936 |
| Cote d'Ivoire Ebola | Complete genome | N/A | N/A |
| virus | | | |
| Marburg virus | Complete genome | Popp | NC_001608 |
| | NP, VP35, VP40, GP, | Popp | Z29337 |
| | VP30, VP24, L genes | | |
| | | Musoke | Z12132 |

### EXAMPLES

### Example 1

### Construction of an Inner Membrane Expression Vector pMPX200

The pMPX-200 expression vector was designed to express a gene product of interest as an inner membrane-bound, periplasmic exposed fusion protein. The nucleotide sequence of this vector is provided as SEQ ID NO: 1 and is shown without a coding sequence of interest. To construct an expression vector containing a coding sequence of interest, one inserts the gene or coding sequence of interest into the SalI/XbaI restriction region of the pMPX200 expression vector to create a chimeric fusion with the transmembrane domain (TMD) of toxR.

### Example 2

### Construction of an Outer Membrane Expression Vector pMPX201

The pMPX-201 expression vector was designed to express a gene product of interest as an outer membrane-bound, extracellular exposed fusion protein. The nucleotide sequence of this vector is provided as SEQ ID NO: 2 and is shown without a coding sequence of interest. To construct an expression vector containing a coding sequence of interest, one inserts the gene or coding sequence of interest into the XhoI/XbaI restriction region of the pMPX201 expression vector to create a chimeric insertion into lamB.

### Example 3

### ToxR-Bacillus anthracis PA Fusion Protein

Immunogenic minicells expressing the soluble Bacillus anthracis protective antigen (PA) on the outer membrane of an E. coli derived minicell are prepared and purified according to the protocols discussed in Examples 1 and 3, E. coli derived minicells transformed with the expression vector discussed in Example 1 but lacking the ToxR::PA fusion protein coding sequence are also prepared as a negative control. The two species of minicells are then formulated for intramuscular injection.

Minicells displaying the ToxR::PA fusion protein are provided to a group of test subjects. On day 1 the test subjects are provided with an initial dose of an inoculum comprising the ToxR::PA fusion protein expressing minicells. Control subjects are provided with an initial dose of an inoculum comprising minicells that do not express the ToxR::PA fusion protein. A booster is provided to each group of animals approximately two weeks later. Approximately 14 days after the initial immunization, blood is taken from the test and control subjects and used in an ELISA to determine if the subjects have mounted an antibody response against the inoculum. Serum analyzed by ELISA indicates that the test subjects mount an antibody response against the fusion protein inoculum. Serum taken from the control animals indicates that while the animals mounted an antibody response against the minicells themselves, they have not produced antibodies that react with the ToxR::PA fusion protein.

Lymphocytes isolated from the test groups of animals are shown to be reactive with the fusion protein expressing minicells. Lymphocytes isolated from the control group animals are shown not to be reactive with the fusion protein expressing minicells.

Approximately three weeks after the initial inoculation, the test animals are challenged with live Bacillus anthracis. The test subjects are completely protected from the development of anthrax symptoms while the control subjects die from anthrax.

What is claimed is:

1. A method of preparing an immunogenic minicell comprising:
preparing an inducible expression vectors wherein the inducible expression vector comprises a heterologous nucleotide sequence encoding an open reading frame of an antigen of interest;
introducing the expression vector to an inducible minicell producing parent cell;
inducing minicell formation from the minicell producing parent cell;
inducing expression of the open reading from of the antigen of interest; and
purifying minicells from the inducible minicell producing parent cell.

2. The method of claim 1, wherein the expression vector comprises the heterologous nucleotide sequence encoding the open reading frame of an antigen of interest operably linked to a nucleotide sequence encoding a transmembrane protein.

3. The method of claim 1, wherein the open reading frame of an antigen of interest encodes a transmembrane protein.

4. The method of claim 2, wherein the transmembrane protein is expressed on the outer membrane of the minicell.

5. The method of claim 2, wherein the transmembrane protein is expressed on the inner membrane of the minicell.

6. The method of claim 1, wherein the immunogenic minicell is derived from a Gram-negative bacterial parent cell.

7. The method of claim 6, wherein the Gram-negative bacteria is selected from the group consisting of Campylobacter jejuni, S. dysenteriae, Lactobacillus spp., Neisseria gonorrhoeae, Legionella Pneumophila, Salmonella spp., Shigella flexneri, and Escherichia coli.

8. The method of claim 1, wherein the immunogenic minicell is derived from a Gram-positive bacterial parent cell.

9. The method of claim 8, wherein the Gram-positive bacteria is selected from the group consisting of Staphylococcus spp., Streptococcus spp., Bacillus subtilis and Bacillus cereus.

10. The method of claim 1, wherein the open reading frame of the antigen of interest is derived from a Bacillus anthracis genome.

11. The method of claim 10, wherein the open reading frame of the antigen of interest encodes a protective antigen with an accession number selected from the group consisting of NC_{—}001496, NC_{—}003980, AF306783, AF306782, AF306781, AF306780, and AF306779.

12. The method of claim 1, wherein the open reading frame of the antigen of interest is derived from a Clostridium botulinum genome.

13. The method of claim 12, wherein the open reading frame of the antigen of interest encodes a protective antigen with an accession number selected from the group consisting of M27892, AY166872, AF464912, AF295926, AF300469, AF300468, AF300467, AF300466, AF300465, D49440, X62389, D90210, D88982, D63903, X54254, AB082519, X70815, X70818, X62683, X62089, Y10770, X70821, X70820, X70816, M92906, AX608812, and X74162.

14. The method of claim 1, wherein the open reading frame of the antigen of interest is derived from a Yersinia pestis genome.

15. The method of claim 14, wherein the open reading frame of the antigen of interest encodes a protective antigen with an accession number selected from the group consisting of X61996, AF053945, NC_{—}003131, AF167310, AF167309, NC_{—}003131, AF074612, and AF053946.

16. A eubacterial minicell comprising a heterologous antigen of interest wherein the antigen of interest is derived from a genome of a pathogen selected from the group consisting of Bacillus anthracis (anthrax), Clostridium botulinum (Botulism), Yersinia pestis, Variola major (smallpox), Francisella tularensis (tularemia), LCM virus, junin virus, machup virus, guanarito virus, lassa fever virus, bunyavirus, hantaviruse, rift valley fever virus, dengue virus, ebola virus, and marburg virus.

17. The eubacterial minicell of claim 16, wherein the transmembrane protein is expressed on the outer membrane of the minicell.

18. The eubacterial minicell of claim 16, wherein the transmembrane protein is expressed on the inner membrane of the minicell.

19. The eubacterial minicell of claim 16, wherein the immunogenic minicell is derived from a Gram-negative bacterial parent cell.

20. The Eubacterial minicell of claim 19, wherein the Gram-negative bacteria is selected from the group consisting of Campylobacter jejuni, S. dysenteriae, Lactobacillus spp., Neisseria gonorrhoeae, Legionella Pneumophila, Salmonella spp., Shigella flexneri, and Escherichia coli.

21. The eubacterial minicell of claim 16, wherein the immunogenic minicell is derived from a Gram-positive bacterial parent cell.

22. The eubacterial minicell of claim 21, wherein the Gram-positive bacteria is selected from the group consisting of Staphylococcus spp., Streptococcus spp., Bacillus subtilis and Bacillus cereus.

23. The eubacterial minicell of claim 16, wherein the open reading frame of the antigen of interest is derived from a Bacillus anthracis genome.

24. The eubacterial minicell of claim 23, wherein the antigen of interest is encoded by a polynucleotide having an accession number selected from the group consisting of NC_{—}001496, NC_{—}003980, AF306783, AF306782, AF306781, AF306780, and AF306779.

25. The eubacterial minicell of claim 16, wherein the open reading frame of the antigen of interest is derived from a Clostridium botulinum genome.

26. The eubacterial minicell of claim 25, wherein the open reading frame of the antigen of interest encodes a protective antigen with an accession number selected from the group consisting of M27892, AY166872, AF464912, AF295926, AF300469, AF300468, AF300467, AF300466, AF300465, D49440, X62389, D90210, D88982, D63903, X54254, AB082519, X70815, X70818, X62683, X62089, Y10770, X70821, X70820, X70816, M92906, AX608812, and X74162.

27. The eubacterial minicell of claim 16, wherein the open reading frame of the antigen of interest is derived from a Yersinia pestis genome.

28. The eubacterial minicell of claim 27, wherein the open reading frame of the antigen of interest encodes a protective antigen with an accession number selected from the group consisting of X61996, AF053945, NC_{—}003131, AF167310, AF167309, NC_{—}003131, AF074612, and AF053946.
